(19) United States
(12) Patent Application Publication
(10) Pub. No.: US 2008/0071236 A1
(43) Pub. Date: Mar. 20, 2008

(54) Combinative assembly for an artificial anus

(76) Inventor: Wen-Hao Lee, Fongyuan City (TW)

Correspondence Address: Frenkel & Associates, P.C., Suite 330, 3975 University Drive, Fairfax, VA, 22030, US

(21) Appl. No.: 11/521,898

(22) Filed: Sep. 15, 2006

(51) Int. Cl. A61F 5/44

(52) U.S. Cl. 604/333; 604/338

(57) ABSTRACT

A combination assembly for an artificial anus has a spacer, a connecting panel and a mounting bracket. The spacer contacts with the skin of the human body and has a mounting hole formed through the spacer. The connecting panel is attached to the spacer and has a body. The body has a mounting hole, two mortises communicated with the mounting hole, and two mounting grooves communicated with the mortises. The mounting bracket is connected to the body and the spacer and has a mounting tube extended through the mounting holes in the connecting panel and the spacer and two tenons engaged with the mounting grooves respectively, and a bag is mounted around the mounting tube.

### BACKGROUND OF THE INVENTION

1. Field of the Invention

The present invention relates to a combination assembly, and more particularly relates to a combination assembly that used on an artificial anus that is secure and comfortable in use.

2. Description of Related Art

When a person gets carcinoma of the rectum or a traffic accident, he or she needs to have a surgical operation with separating the gut and the anus and connecting the gut to an opening defined in the skin of the person to an artificial anus. Accordingly, patients can excrete the excrements from the artificial anus.

With reference to FIG. 7, a conventional combination assembly (50) for an artificial anus has a bag (51) and an adhesive tape (52). The bag (51) has an open end that communicates with the opening in the skin. The adhesive tape (52) is connected to the bag (51) near the open end and is adhered to the skin around the opening to align and communicate the open end of the bag (51) with the artificial anus.

When patients use the conventional combination assembly (50) with the artificial anus, they need to tear off the adhesive tape (52) and clean the bag (51) or change a new bag (51) with an adhesive tape (52).

With reference to FIGS. 8 and 9, another conventional combination assembly (60) for an artificial anus has a connecting panel (61), a mounting bracket (62) and a bag (63). The connecting panel (61) contacts with the skin of the human body around the opening and has a front side, a rear side, a mounting hole (611), two mortises (612) and two mounting grooves (613).

The mounting hole (611) is formed from the front side to the rear side of the connecting panel (61) and is communicated with the opening. The mortises (612) are formed through the connecting panel (61) and are communicated with the mounting hole (611). The mounting grooves (613) are formed in the rear side of the connecting panel (61) and communicates with the mortises (612) respectively.

The mounting bracket (62) is connected detachable to the connecting panel (61) and has a proximal end, a distal end, a mounting tube (621), two tenons (622), a head (623), a draining hole (624) and a retaining ring (625). The mounting tube (621) is hollow, is formed in the proximal end of the mounting bracket (62), is extended through the mounting hole (611) of the connecting panel (61) and has an external surface.

The tenons (622) are formed on and protruded up from the external surface of the mounting tube (621), correspond respectively to the mortises (612) and are engaged with the mounting grooves (613) respectively by rotating the mounting bracket (62) relative to the connecting panel (61).

The head (623) is formed on the distal end of the mounting bracket (62). The draining hole (624) is formed through the head (623) and communicates with the mounting tube (621). The retaining ring (625) is formed around the mounting bracket (62) between the mounting tube (621) and the head (623).

With reference to FIG. 9, the bag (63) is mounted around the mounting bracket (62) between the retaining ring (625) and the mounting tube (621)to store the excrements from the artificial anus.

However, the conventional combination assembly (50, 60) has the following shortcomings.

1. When patients use the first conventional combination assembly (50) with the artificial anus, he or she needs to adhere and tear off the adhesive tape (52) from the skin. This is inconvenient in use and may cause the open end of the bag (51) not aligning with the opening. Furthermore, adhering the adhesive tape (52) to the skin of the human body for a long time will cause patients feel sultry or hypersensitive.

2. When patients use the second conventional combination assembly (60) with the artificial anus, the mounting tube (621) is extended through the mounting hole (611) and is pressed against the skin of the human body around the opening. However, this may cause users feel uncomfortable. In addition, patients may adjust the conventional combination assembly (60) when they feel uncomfortable, but this may cause the draining hole (624) of the mounting bracket (62) mis-aligning with the opening and the excrements may excrete out of the conventional combination assembly (60).

Therefore, the present invention provides a combination assembly for an artificial anus to mitigate or obviate the aforementioned problems.

### SUMMARY OF THE INVENTION

The main objective of the present invention is to provide a combination assembly for an artificial anus, which can be assembled easily and quickly with the artificial anus and comfortable in use.

The combination assembly for an artificial anus has a spacer, a connecting panel and a mounting bracket. The spacer contacts with the skin of the human body and has a mounting hole formed through the spacer. The connecting panel is attached to the spacer and has a body. The body has a mounting hole, two mortises communicated with the mounting hole, and two mounting grooves communicated with the mortises. The mounting bracket is connected to the body and the spacer and has a mounting tube extended through the mounting holes in the connecting panel and the spacer and two tenons engaged with the mounting grooves respectively, and a bag is mounted around the mounting tube.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a combination assembly for an artificial anus in accordance with the present invention;

FIG. 2 is another perspective view of the combination assembly in FIG. 1;

FIG. 3 is an enlarged exploded perspective view of the combination assembly in FIG. 1;

FIG. 4 is an enlarged operational cross sectional side view in partial section of the combination assembly in FIG. 1;

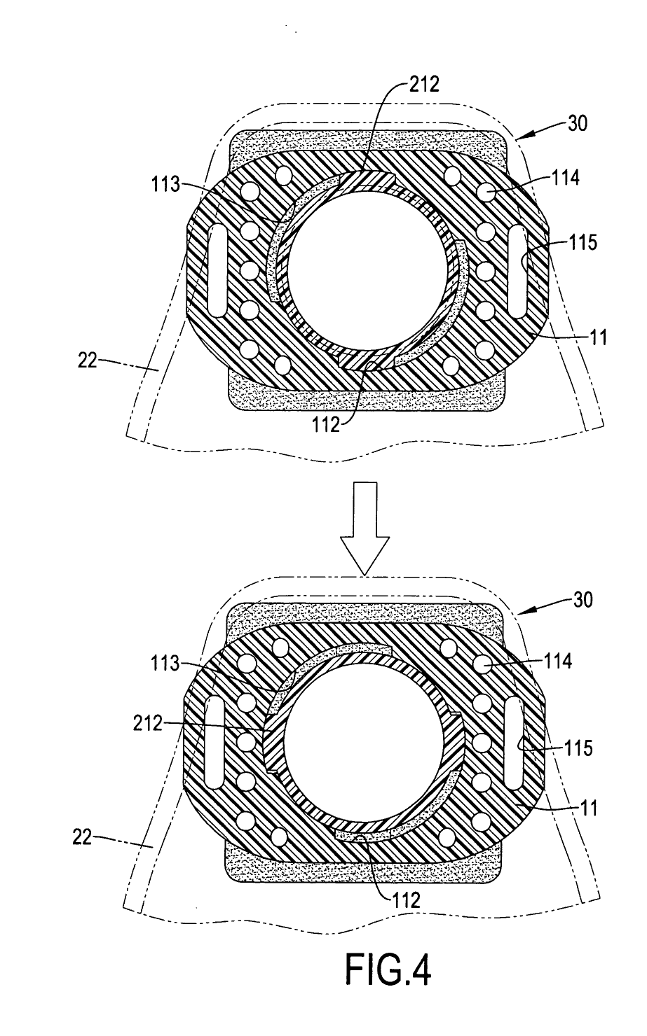

FIG. 5 is an enlarged side view in partial section of the combination assembly in FIG. 1;

FIG. 6 is a perspective view of another embodiment of a combination assembly for an artificial anus in accordance with the present invention;

FIG. 7 is an operational perspective view of a first conventional combination assembly for an artificial anus in accordance with the prior art;

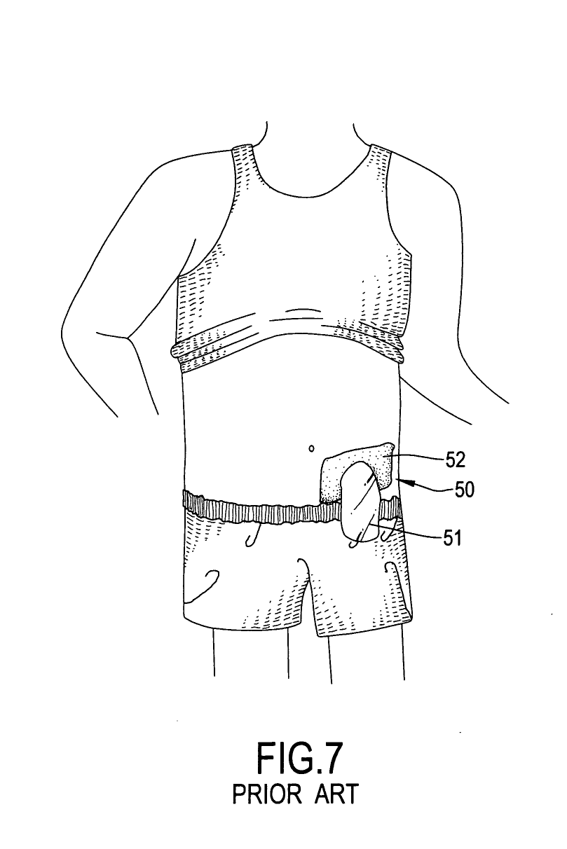

FIG. 8 is an exploded perspective view of a second conventional combination assembly for an artificial anus in accordance with the prior art; and

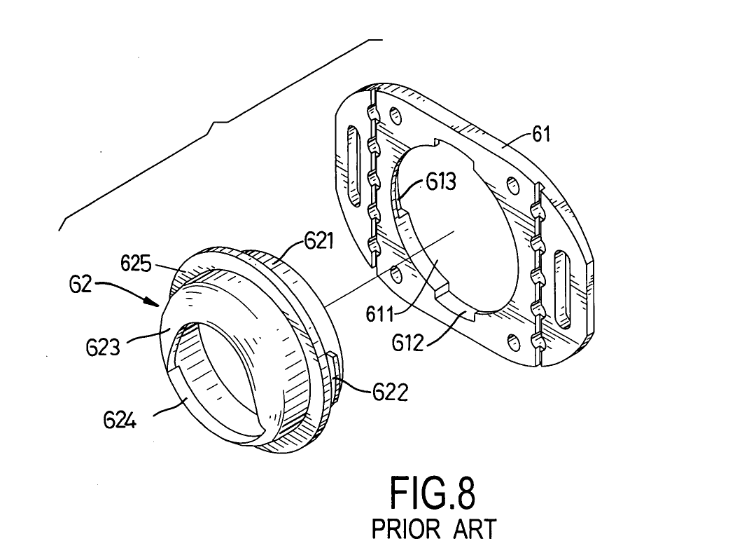

FIG. 9 is a side view in partial section of the conventional combination assembly in FIG. 8.

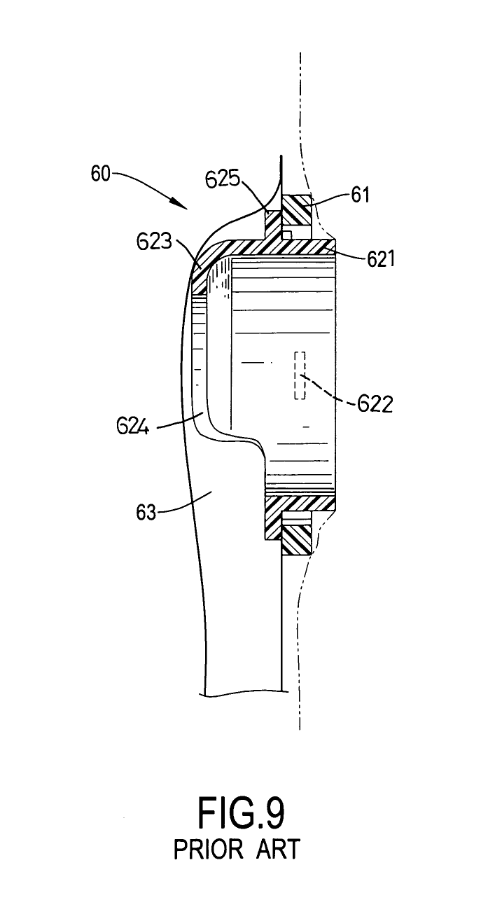

### DETAILED DESCRIPTION OF THE PREFERRED EMBODIMENT

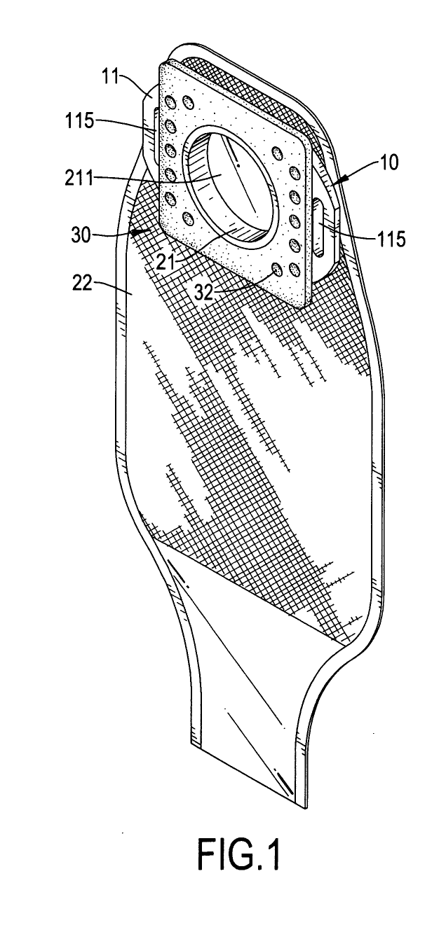

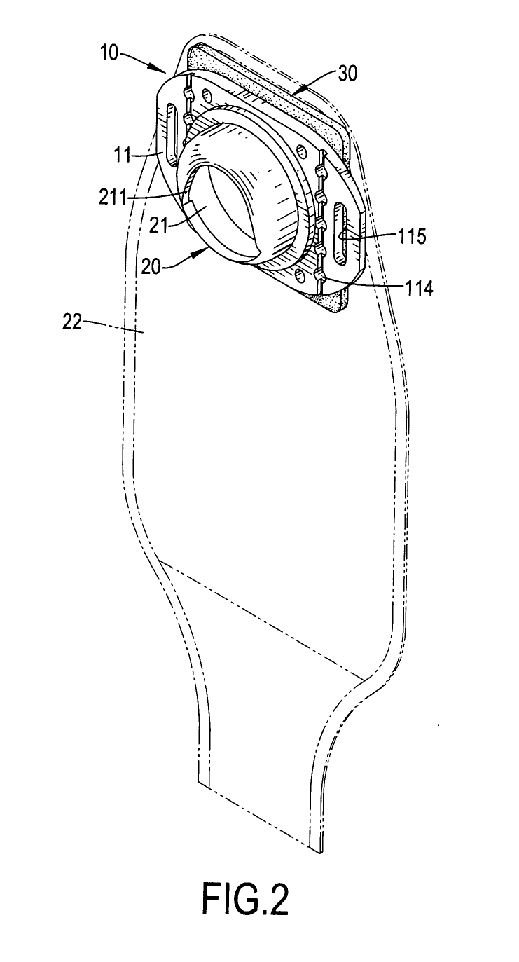

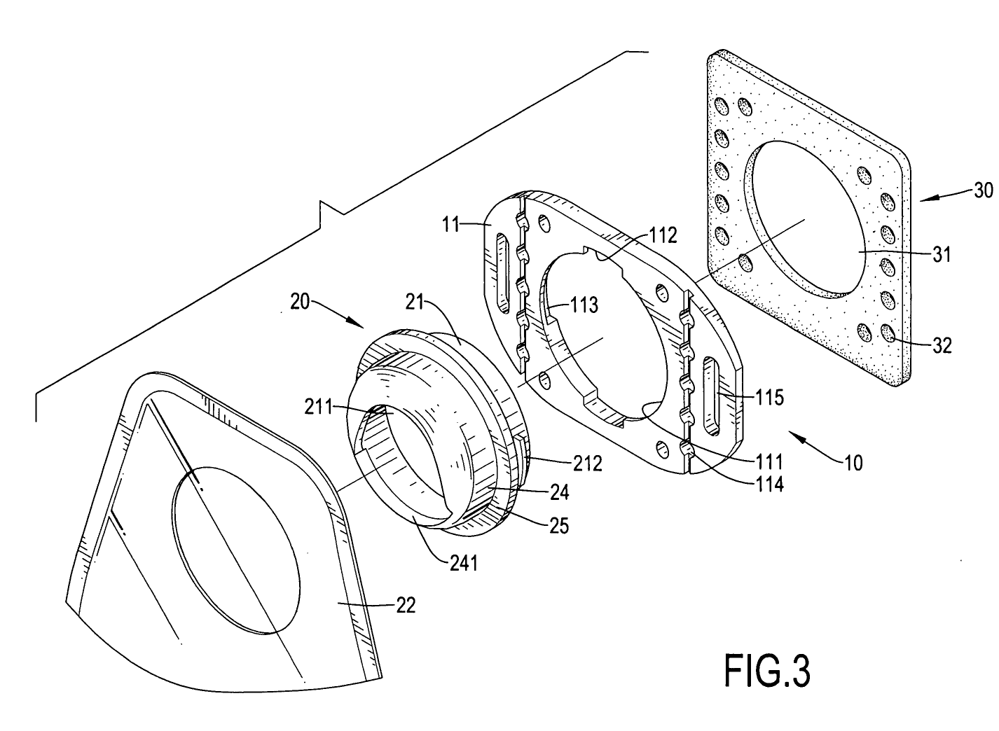

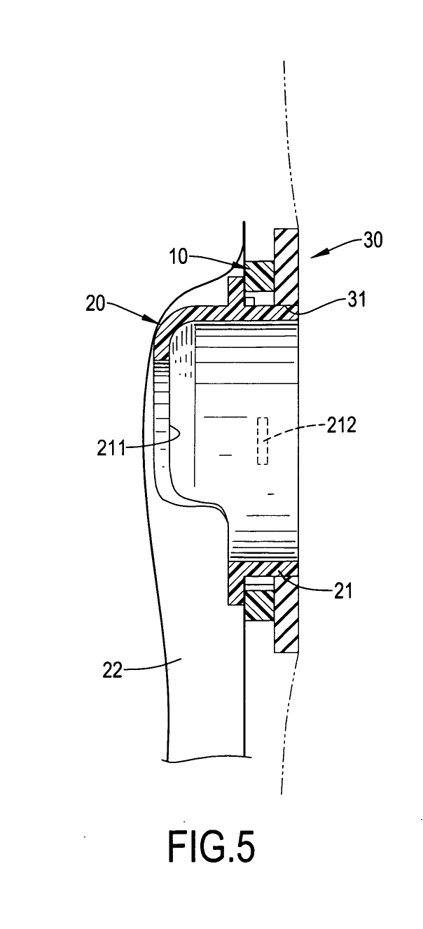

With reference to FIGS. 1 to 3 and 5, a combination assembly for an artificial anus with an opening in accordance with the present invention comprises a spacer (30), a connecting panel (10) and a mounting bracket (20).

The spacer (30) may be made of soft silica gel, is contacted with the skin of the human body and has a rear side, a front side, a mounting hole (31) and multiple through holes (32). The rear side of the spacer (30) contacts with the skin of the human body around the opening. The mounting hole (31) is formed from the front side to the rear side of the spacer (30) and communicates with the opening. The through holes (32) are formed through the spacer (30) around the mounting hole (31).

The connecting panel (10) may be made of Polyurethane, is contacted with the front side of the spacer (30) and has a body (11) and an optional belt (12). The body (11) is attached to the spacer (30) and has a rear side, a front side, two ends, a mounting hole (111), two mortises (112), two mounting grooves (113), multiple ventilative holes (114) and two connecting holes (115).

The rear side of the body (11) is attached to the front side of the spacer (30). The mounting hole (111) is formed from the front side to the rear side of the body (11), is communicated with the mounting hole (31) in the spacer (30) and the opening of the artificial anus. The mortises (112) are formed through the body (11) and communicate with the mounting hole (11). The mounting grooves (113) are formed in the rear side of the body (11) and communicate with the mortises (112) respectively.

The ventilative holes (114) are formed through the body (11) and communicate with the through holes (32) in the spacer (30) to provide a ventilating effect to the spacer (30) and the connecting panel (10). The connecting holes (115) are formed through the body (11) near the ends respectively.

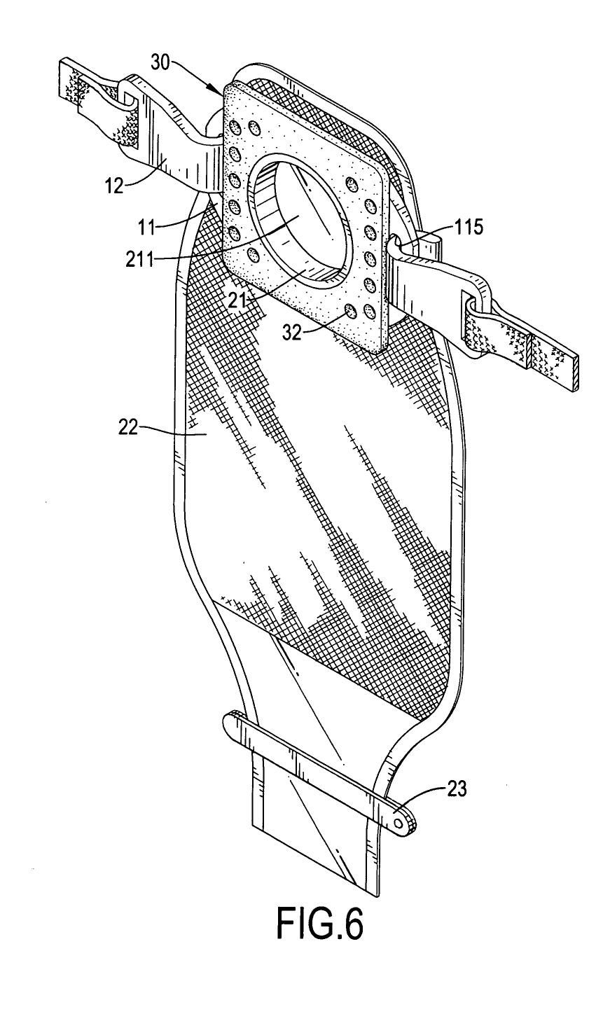

With reference to FIG. 6, the belt (12) is attached to the body (11), is used to mount the connecting panel (10) with the skin of the human body with two paws. The paws are respectively attached to the connecting holes (115) in the body (11).

The mounting bracket (20) may be made of Polyurethane, is connected detachable to the body (11) of the connecting panel (10) and the spacer (30) and has a proximal end, a distal end, a mounting tube (21), a head (24), a retaining ring (25), a bag (22) and a clamp (23).

The mounting tube (21) is formed on the proximal end of the mounting bracket (20), is extended through the mounting holes (111) of the connecting panel (10) and the spacer (30) and has an external surface, a central hole (211) and two tenons (212).

The central hole (211) is formed through the mounting tube (21) and is communicated with the mounting holes (111) in the connecting panel (10)and the spacer (30) and the opening of the artificial anus. The tenons (212) are formed on and protruded up from the external surface of the mounting tube (21), correspond respectively to the mortises (112) and are engaged with the mounting grooves (113) respectively by rotating the mounting bracket (20) with the body (10) of the connecting panel (10).

The head (24) is formed on the distal end of the mounting bracket (20) and has a draining hole (241). The draining hole (241) is formed through the head (24) and communicates with the central hole (211) of the mounting tube (21). The retaining ring (25) is formed around the mounting bracket (20) between the mounting tube (21) and the head (24).

The bag (22) is mounted around the mounting bracket (20) to store the excrements from the opening of the artificial anus and has a sidewall, a lower end, a covering hole and an opening. The covering hole is formed in the sidewall of the bag (22) and is mounted around the head (24) between the retaining ring (25) and the tenons (212) on the mounting tube (21). With reference to FIG. 6, the opening is formed in the lower end of the bag (22) oppositely the covering hole. The clamp (23) is clipped with the bag (22) near the opening.

The combination assembly for an artificial anus has the following advantages.

1. The spacer (30) is made of soft silica gel and contacts with the skin of the human body, this can prevent the mounting bracket (20) directly pressed against the human body and keep the mounting bracket (20) and the connecting panel (10) at a position exactly around the opening.

2. The through holes (32) in the spacer (30) and the ventilative holes (114) in the body (11) of the connecting panel (10) can allow the air flowing through the spacer (30) and the connecting panel (10) and prevent patient feeling sultry or hypersensitive.

3. The spacer (30) contacting with the skin of the human body can prevent the excrements from excreting out of the combination assembly.

4. Patients can clean up the excrements easily and conveniently by changing a new mounting bracket (20) with a new bag (22) or unlocking the clamp (23) to open the opening of the bag (22).

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A combination assembly for an artificial anus in a human body with an opening in a skin, and the combination assembly comprising
a spacer being adapted to contact with the skin and having
a rear side being adapted to contact with the skin of the human body around the opening;
a front side; and
a mounting hole being formed from the front side to the rear side of the spacer and being adapted to communicate with the opening;
a connecting panel being attached to the front side of the spacer and having
a body being attached to the spacer and having
a rear side being attached to the front side of the spacer;
a front side;
a mounting hole being formed from the front side to the rear side of the body and being communicated with the mounting hole in the spacer;
two mortises being formed through the body and communicated with the mounting hole in the body; and
two mounting grooves being formed in the rear side of the body and being communicated with the mortises respectively; and
a mounting bracket being connected detachable to the body of the connecting panel and the spacer and having
a proximal end;
a distal end;
a mounting tube being formed on the proximal end of the mounting bracket, being extended through the mounting holes of the connecting panel and the spacer and having
an external surface;
a central hole being formed through the mounting tube, being communicated with the mounting holes in the connecting panel and the spacer; and
two tenons being formed on and protruded up from the external surface of the mounting tube, corresponding respectively to the mortises and being engaged with the mounting grooves respectively by rotating the mounting bracket relative to the body of the connecting panel; and
a bag being mounted around the mounting tube of the mounting bracket.

2. The combination assembly for an artificial anus as claimed in claim 1, wherein
the mounting bracket further has
a head being formed on the distal end of the mounting bracket with a draining hole formed through the head and communicated with the central hole of the mounting tube; and
a retaining ring being formed around the mounting bracket between the mounting tube and the head; and
the bag further has
a sidewall; and
a covering hole being formed in the sidewall of the bag and being mounted around the head between the retaining ring and the tenons in the mounting tube.

3. The combination assembly for an artificial anus as claimed in claim 2, wherein
the spacer further has multiple through holes being formed through the spacer around the mounting hole; and
the body of the connecting panel further has multiple ventilative holes being formed through the body and being communicated with the through holes in the spacer.

4. The combination assembly for an artificial anus as claimed in claim 3, wherein
the body has
two ends; and
two connecting holes being formed through the body near the ends respectively; and
the connecting panel further has a belt being attached to the body with two paws attached to the connecting holes.

5. The combination assembly for an artificial anus as claimed in claim 4, wherein
the bag has
a lower end; and
an opening being formed in the lower end of the bag oppositely the covering hole; and
the mounting bracket further has a clamp being clipped with the bag near the opening.
